(19) United States
(12) Patent Application Publication
(10) Pub. No.: US 2009/0187247 A1
(43) Pub. Date: Jul. 23, 2009

(54) Spinal implant having a resorbable anchor device for temporarily securing an interbody device to adjacent upper and lower vertebrae

(76) Inventors: Newton H. Metcalf, JR., Memphis, TN (US); Greg C. Marik, Memphis, TN (US); Kevin T. Foley, Germantown, TN (US)

Correspondence Address: MEDTRONIC;Attn: Noreen Johnson - IP Legal Department, 2600 Sofamor Danek Drive, MEMPHIS, TN, 38132, US

(21) Appl. No.: 12/009,988

(22) Filed: Jan. 23, 2008

(51) Int. Cl. A61F 2/44; A61B 17/56

(52) U.S. Cl. 623/17.16; 623/17.11; 606/301

(57) ABSTRACT

A spinal implant including an interbody device and an anchor device. The interbody device is sized and shaped to be positioned within an intervertebral disc space between adjacent upper and lower vertebrae. The anchor device is at least partially formed of a resorbable material and is coupled to the interbody device and anchored to the upper and lower vertebrae. The anchor device is configured to temporarily secure the interbody device to the upper and lower vertebrae for a period of time sufficient to facilitate bone growth onto and/or through the interbody device and is thereafter resorbed into the body.

### FIELD OF THE INVENTION

The present invention relates generally to spinal implants for use in association with treatment of the spinal column, and more particularly relates to an interbody device temporarily secured to adjacent vertebrae via a resorbable anchor device.

### BACKGROUND

A variety of intervertebral implants are available for disc replacement and spinal fusion procedures. These implants are typically formed from various materials including stainless steel, titanium, composites, allograft, xenograft or other biocompatible materials, and have the necessary strength to prevent the intervertebral disc space from collapsing.

One disadvantage with some prior art intervertebral implants is that the implant or other material placed within the intervertebral disc space might move or migrate, thereby creating a risk of expulsion or dislodgement from the disc space. Expulsion or dislodgement of the implant may in turn result in the possible collapse of the disc space and/or damage or trauma to adjacent soft tissues or neural structures. While threaded implants and implants having integral anchoring elements (e.g., teeth, spikes, or other surface protrusions) can provide improved migration-resistant capabilities, insertion of such implants into the disc space can be difficult, time-consuming, and may require over distraction of the intervertebral disc space. Additionally, some implants are anchored to the adjacent vertebrae via anchor devices (e.g., bone screws) and/or various types of elongate elements that extend across the intervertebral disc space to prevent expulsion or dislodgement of the fusion device from the disc space. However, such anchor devices and elongate elements may have to be removed in a subsequent surgical procedure and/or may interfere with the subsequent implantation of another fusion device within an adjacent intervertebral disc space and anchoring of the fusion device to the adjacent vertebrae.

Thus, there remains a need for an improved spinal implant including features for temporary fixation to adjacent vertebrae. The present invention satisfies this need and provides other benefits and advantages in a novel and unobvious manner.

### SUMMARY

The present invention relates generally to a spinal implant for use in association with treatment of the spinal column, and more particularly relates to an interbody device temporarily secured to adjacent vertebrae via a resorbable anchor device. While the actual nature of the invention covered herein can only be determined with reference to the claims appended hereto, certain forms of the invention that are characteristic of the preferred embodiments disclosed herein are described briefly as follows.

In one form of the present invention, a spinal implant is provided including an interbody device sized and shaped to be positioned within an intervertebral disc space between adjacent upper and lower vertebrae. The spinal implant also includes an anchor device at least partially formed of a resorbable material and coupled to the interbody device and anchored to the upper and lower vertebrae. The anchor device is configured to temporarily secure the interbody device to the upper and lower vertebrae for a period of time sufficient to facilitate bone growth onto and/or through the interbody device.

In another form of the present invention, a spinal implant is provided including an interbody device sized and shaped to be positioned within an intervertebral disc space between adjacent upper and lower vertebrae. The spinal implant also includes an elongate member formed of a resorbable material and having a length sized to span a distance between the upper and lower vertebrae. The elongate member is coupled to the interbody device and has first and second end portions, with the first end portion anchored to the upper vertebrae by a first bone engaging member, and the second end portion anchored to the lower vertebrae by a second bone engaging member. The elongate member is configured to temporarily secure the interbody device to the upper and lower vertebrae for a period of time sufficient to facilitate bone growth onto and/or through the interbody device.

In another form of the present invention, a method of vertebral interbody fusion includes providing an interbody device and an anchor device at least partially formed of a resorbable material, positioning the interbody device within an intervertebral disc space between adjacent upper and lower vertebrae, coupling the anchor device with the interbody device, engaging the anchor device to the upper and lower vertebrae, and wherein the anchor device temporarily secures the interbody device to the upper and lower vertebrae for a period of time sufficient to facilitate bone growth onto and/or through the interbody device.

It is one object of the present invention to provide an improved spinal implant including features for temporary fixation to adjacent vertebrae. Further objects, features, advantages, benefits, and aspects of the present invention will become apparent from the drawings and description contained herein.

### BRIEF DESCRIPTION OF THE DRAWINGS

FIG. 1 is a perspective view of a spinal implant according to one form of the present invention.

FIG. 2 is a side view of a portion of the spinal column with the spinal implant shown in FIG. 1 secured to adjacent vertebrae via a resorbable anchor device.

FIG. 3 is a side view of a spinal implant according to another form of the present invention.

FIG. 4 is a side view of a spinal implant according to another form of the present invention.

FIG. 5 is a side view of a spinal implant according to another form of the present invention.

FIG. 6 is a side view of a portion of the spinal column with a spinal implant according to another form of the present invention secured to adjacent vertebrae via a resorbable anchor device.

FIG. 7 is a side view of a portion of the spinal column with a spinal implant according to another form of the present invention secured to adjacent vertebrae via a resorbable anchor device.

FIG. 8 is a perspective view of another embodiment of the spinal implant illustrated in FIG. 1 including a solid, porous interbody device and a resorbable anchor device.

### DESCRIPTION OF THE PREFERRED EMBODIMENTS

For the purposes of promoting an understanding of the principles of the invention, reference will now be made to the embodiments illustrated in the drawings and specific language will be used to describe the same. It will nevertheless be understood that no limitation of the scope of the invention is hereby intended, and that alterations and further modifications to the illustrated devices and/or further applications of the principles of the invention as illustrated herein are contemplated as would normally occur to one skilled in the art to which the invention relates.

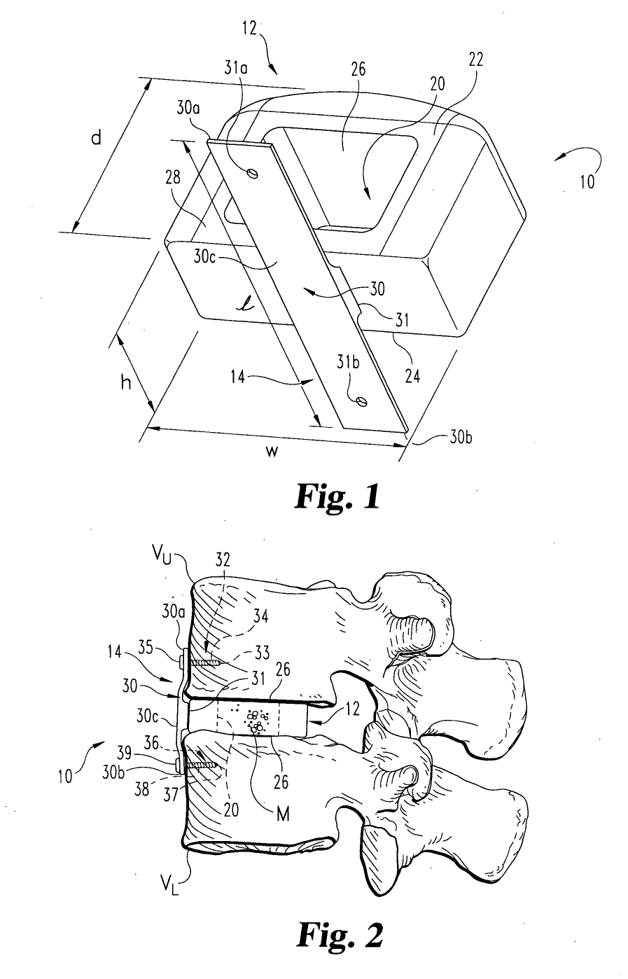

Referring to FIGS. 1 and 2, shown therein is a spinal implant 10 according to one form of the present invention. The spinal implant 10 generally includes an interbody device 12 sized and shaped to be positioned within an intervertebral disc space between adjacent upper and lower vertebrae V_{U}, V_{L}, and an anchor device 14 at least partially formed of a bioresorbable material and configured to temporarily secure the interbody device 12 to the adjacent upper and lower vertebrae V_{U}, V_{L} for a period of time sufficient to facilitate bone growth between the adjacent vertebrae V_{U}, V_{L} and the interbody device 12. In one embodiment of the invention, the upper and lower vertebrae V_{U}, V_{L} and the disc space are accessed via an anterior surgical approach using known surgical techniques. However, other surgical approaches, including posterior and lateral approaches, are also contemplated. In a further embodiment of the invention, the disc material may be removed from the disc space and the disc space height restored, if necessary, using known surgical techniques to prepare the disc space for receipt of the interbody device 12. In one embodiment of the invention, the spinal implant 10 is used in association with the cervical region of the spine. However, it should be understood that the spinal implant 10 may be used in association with other regions of the spine, including the thoracic or lumbar regions of the spine. Further details regarding the spinal implant 10 and its components are set forth below.

In one embodiment of the invention, the interbody device 12 has a height h which substantially corresponds to the natural disc space height between the adjacent vertebrae V_{U}, V_{L}, and a depth d and a width w which substantially correspond to the outer profile of the natural vertebral endplates such that the interbody device 12 substantially fills the intervertebral disc space between the adjacent vertebrae V_{U}, V_{L}. Although the interbody device 12 is illustrated as having a substantially uniform height h, it should be understood that the interbody device 12 may be configured to have a tapering height to accommodate for the lordotic angle between the endplates of the adjacent upper and lower vertebrae V_{U}, V_{L}. The interbody device 12 can be made from any bio-compatible material including, for example, titanium, surgical stainless steel, metallic alloys such as a titanium alloy, polymeric materials such as PEEK, composite materials such as carbon composites, ceramic materials, bone or bone substitute materials, porous materials to facilitate bone in-growth, or any other suitable bio-compatible material known to those of skill in the art. The interbody device 12 is preferably formed of a substantially rigid, non-resorbable material to provide sufficient structural integrity to resist loadings exerted onto the interbody device 12 by the adjacent vertebrae V_{U}, V_{L}.

In the illustrated embodiment, the interbody device 12 is configured as a fusion device that promotes fusion between the upper and lower vertebrae V_{U}, V_{L}. The illustrated embodiment of the interbody device 12 includes a hollow interior chamber 20 and upper and lower vertebral engaging surfaces 22, 24 configured for engagement with vertebral endplates of the upper and lower vertebrae V_{U}, V_{L}. Each of the upper and lower vertebral engaging surfaces 22, 24 defines at least one bone growth opening 26 extending therethrough and communicating with the hollow interior chamber 20. In one embodiment, the upper and lower vertebral engaging surfaces 22, 24 are substantially flat and smooth. However, in other embodiments, the vertebral engaging surfaces 22, 24 may be provided with a plurality of surface features that engage the vertebral endplates to inhibit movement of the fusion device, including, for example, teeth or peeks, ridges, grooves or valleys, recessed regions, spikes, surface roughening, or other suitable surface features that would occur to one of skill in the art. In the illustrated embodiment, the interbody device 12 includes a continuous outer parametrical wall 28 which defines the hollow interior chamber 20, the upper and lower vertebral engaging surfaces 22, 24, and the bone growth openings 26.

Although a particular type and configuration of the interbody device 12 has been illustrated and described herein, it should be understood that other types and configurations of fusion devices are also contemplated for use in association with the present invention, including, for example, threaded and non-threaded fusion cages and various configurations of fusion devices having alternative shapes and sizes. It should also be understood that other types of interbody devices are also contemplated for use in association with the present invention including, for example, intervertebral spacers, motion preserving devices including artificial discs and articulating implants, expandable implants, bone graft implants, vertebral replacement devices, corpectomy devices or any other type of interbody implant that would occur to one of skill in the art. Additionally, although the illustrated embodiment of the invention depicts a single interbody device 12 positioned within the disc space and temporarily secured to the upper and lower vertebrae V_{U}, V_{L} via the resorbable anchor device 14, it should be understood that in other embodiments of the invention, a pair of interbody devices may be bilaterally positioned within the disc space.

In one embodiment of the invention, a bone growth material M may be positioned within the hollow interior chamber 20 to promote bone growth from the upper and lower vertebrae V_{U}, V_{L} and into the hollow interior chamber 20 via the bone growth openings 26. The bone growth preferably extends entirely through the interbody device 12 to provide a solid mass of bony material extending from the upper vertebra V_{U} to the lower vertebra V_{L}. The bone growth material M is preferably positioned within the hollow interior chamber 20 prior to insertion of the interbody device 12 with the disc space between the upper and lower vertebrae V_{U}, V_{L}. However, it should be understood that the interbody device 12 may be configured to allow for loading of the bone growth material M into the hollow interior chamber 20 subsequent to insertion into the disc space. The bone growth material M can be any type of material that promotes bone growth, including, for example, bone morphogenic protein (BMP), bone graft material, bone chips or bone marrow, demineralized bone matrix (DBM), mesenchymal stem cells, LIM mineralization protein (LMP), or any other suitable bone growth promoting material or substance known to those of skill in the art. Further, the bone growth material M may be used with or without a suitable carrier.

As indicated above, the anchor device 14 is at least partially formed of a bioresorbable material and may be operably coupled to the interbody device 12. In one embodiment, the anchor device 14 is engaged to the upper and lower vertebrae V_{U}, V_{L} and is configured to temporarily secure the interbody device 12 to the upper and lower vertebrae V_{U}, V_{L} for a period of time sufficient to facilitate bone growth therebetween. In a further embodiment, the anchor device 14 is configured to temporarily rigidly secure the interbody device 12 to the upper and lower vertebrae V_{U}, V_{L} to substantially prevent relative movement therebetween to facilitate bone growth. The bone growth occurs through the bone growth openings 26 in the interbody device 12 and preferably into and through the hollow interior chamber 20. Subsequent to such bone growth, at least a portion of the anchor device 14 is resorbed or absorbed into the body and the force initially exerted onto the interbody device 12 by the anchor device 14 to secure the interbody device 12 in position relative to the upper and lower vertebrae V_{U}, V_{L} is removed. As a result, a subsequent surgical procedure to remove the anchor device 14 from the patient is not required. Further, removal of the force initially exerted onto the interbody device 12 tends to avoid stress shielding of the new bone growth. Moreover, resorption of the anchor device 14 into the body allows for subsequent engagement of additional spinal implants to adjacent vertebral levels in future operations without interference from the previously implanted spinal implant 10. Specifically, the anchor device 14 associated with the previously implanted spinal implant 10 would not interfere with engagement of additional spinal implants to adjacent vertebral levels since the anchor device 14 would have already be resorbed into the body.

As indicated above, the anchor device 14 is at least partially formed of a bioresorbable material. Examples of bioreabsorbable materials include polyglycolate polymers or analogues, lactides, polydioxanone, polyglyconate, lactide/glycolide copolymers, tyrosine-derived polycarbonate, polyanhydride, polyorthoester, polyphosphazene, calcium phosphate, hydroxyapatite, bioactive glass, fibrin, albumin, gelatin, collagen, elastin, silk, demineralized bone matrix, polyethylene oxide, polyethylene glycol, polyvinyl alcohol, polypropylene fumarate, or any combinations thereof. However, other suitable bioresorbable materials are also contemplated as would occur to one of skill in the art. As should be appreciated, selection of the resorbable material from which the anchor device 14 is formed affects the length of time necessary to partially or fully biodegrade the anchor device 14.

In the illustrated embodiment of the invention, the anchor device 14 is configured as an elongate member 30 at least partially formed of a resorbable material and having a length l sized to span a distance between the upper and lower vertebrae V_{U}, V_{L}, with the elongate member 30 anchored to the upper and lower vertebrae V_{U}, V_{L} by at least two bone engaging members 32, 36. In one embodiment, the elongate member 30 is integral with the interbody device 12 and has first and second end portions 30a, 30b and an intermediate portion 30c, with the first end portion 30a anchored to the upper vertebra V_{U} by the first bone engaging member 32, the second end portion 30b anchored to the lower vertebra V_{L} by the second bone engaging member 36, and the intermediate portion 30c operably coupled to the interbody device 12. In the illustrated embodiment, the end portions 30a, 30b of the elongate member 30 are configured as upper and lower flanges extending beyond the upper and lower vertebral engaging surfaces 22, 24 of the interbody device 12. However, other configurations are also contemplated. Additionally, although a single bone engaging member is illustrated for use in anchoring each end portion 30a, 30b of the elongate member 30 to the upper and lower vertebrae V_{U}, V_{L}, it should be understood that two or more bone engaging members may alternatively be used to attach each end portion of the elongate member 30 to the upper and lower vertebrae V_{U}, V_{L}. In the illustrated embodiment of the implant 10, the intermediate portion 30c is slightly enlarged (i.e., has an increased thickness) relative to the remainder of the elongate member 30 so as to define a projecting portion 31 to facilitate placement of the entire interbody device 12 within the disc space between the upper and lower vertebrae V_{U}, V_{L}. Furthermore, the elongate member 30 may also be flexible in order to conform the end portions 30a, 30b to the exterior surfaces of the adjacent vertebrae V_{U}, V_{L}. In the illustrated embodiment of the anchor device 14, the elongate member 30 is provided with at least one opening 31a adjacent the first end portion 30a and at least one opening 31b adjacent the second end portion 30b. The openings 31a, 31b are configured to permit passage of the bone engaging members 32, 36 through the elongate member 30 and into engagement with the upper and lower vertebrae V_{U}, V_{L}. The openings 31a, 31b are illustrated as having a generally circular shape. However, it should be understood that the openings 31a, 31b may take on any suitable shape or configuration that would occur to one of skill in the art, including an elongated slot configuration. Furthermore, in alternative embodiments, the elongate member 30 need not necessarily include the screw-receiving openings 31a, 31b. For example, the bone engaging members 32, 36 may be configured to pierce through or penetrate the elongate member 30. In other alternative embodiments, the bone engaging members need not necessarily pass through the elongate member 30, but may instead be engaged with the upper and lower vertebrae V_{U}, V_{L} at a position adjacent the end portions 30a, 30b, and provided with an enlarged head portion that contacts the end portions 30a, 30b to secure the elongate member 30 to the upper and lower vertebrae V_{U}, V_{L}.

In the illustrated embodiment, the elongate member 30 has a plate-like configuration. However, other types and configurations of elongate members are also contemplated as falling within the scope of the present invention, including, for example, rods, blocks, tethers, wires or any other suitable elongate member known to those of skill in the art. Additionally, in the illustrated embodiment, the bone engaging members 32, 36 are configured as bone screws, with each respectively including a threaded stem portion 34, 38 extending between a leading tip 33, 37 and a head portion 35, 39. It should be appreciated that the elongate member 30 may include one or more recessed regions or countersunk regions surrounding the screw-receiving openings 31a, 31b to receive the screw head portions 35, 39 in a substantially flush relationship with respect to the outwardly facing surface of the elongate member 30 in order to reduce interference with nearby tissue or other anatomic features or structures. Although a particular type and configuration of the bone engaging members 32 and 36 have been illustrated and described for use in association with the implant 10, it should be understood that other types and configurations of bone engaging members are also contemplated as falling within the scope of the present invention, including, for example, tacks, bolts, pins, prongs, hooks, anchors or any other suitable bone engaging member known to those of skill in the art.

In one embodiment of the invention, the entire elongate member 30 is formed of a resorbable material. However, in other embodiments of the invention, only portions of the elongate member 30 are formed of a resorbable material. For example, in one specific embodiment, the end portions 30a, 30b are formed of a resorbable material, with the intermediate portion 30c formed of a non-resorbable material. In another specific embodiment, the intermediate portion 30c may be formed of a resorbable material, with the end portions 30a, 30b formed of a non-resorbable material. In a further embodiment of the invention, the bone engaging members 32, 36 are formed of a non-resorbable material. However, in other embodiments, all or part of the bone engaging members 32, 36 may be formed of a resorbable material that is either the same as or different from the resorbable material from which the elongate member 30 is formed. In one embodiment of the invention, the elongate member 30 of the anchor device 14 is formed integral with the interbody device 12 to form a unitary, single piece structure. However, in other embodiments, the anchor device 14 may be formed separately from the interbody device 12 and coupled thereto via a suitable coupling technique including, for example, via adhering, fusing, screwing, bolting, clamping, pinning, riveting, tacking or any other coupling technique know to those of skill in the art.

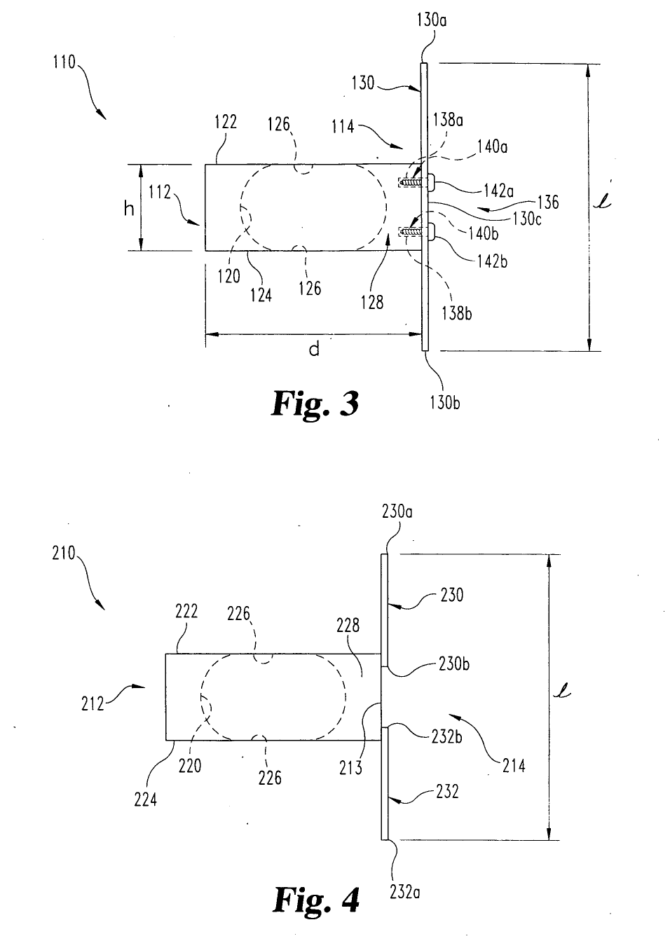

Referring to FIG. 3, shown therein is a spinal implant 110 according to another form of the present invention. The spinal implant 110 generally includes an interbody device 112 sized and shaped to be positioned within an intervertebral disc space between adjacent vertebrae, and an anchor device 114 at least partially formed of a bioresorbable material and configured to temporarily secure the interbody device 112 to the adjacent vertebrae for a period of time sufficient to facilitate bone growth therebetween.

Similar to the interbody device 12 associated with the spinal implant 10, the interbody device 112 generally includes a height h which substantially corresponds to the natural disc space height between the adjacent vertebrae, and a depth d and a width w (not shown) which substantially correspond to the outer profile of the natural vertebral endplates such that the interbody device 112 substantially fills the intervertebral disc space between the adjacent vertebrae. The interbody device 112 may be formed from any of the materials set forth above with regard to the interbody device 12, and may be provided with any of the elements or features associated with the embodiments set forth above with regard to the interbody device 12. In the illustrated embodiment, the interbody device 112 is configured as a fusion device that promotes fusion between the adjacent vertebrae, and includes a hollow interior chamber 120 and upper and lower vertebral engaging surfaces 122, 124 configured for engagement with the endplates of adjacent vertebrae. Each of the upper and lower vertebral engaging surfaces 122, 124 defines at least one bone growth opening 126 extending therethrough and communicating with the hollow interior chamber 120. In the illustrated embodiment, the interbody device 112 includes a continuous outer parametrical wall 128 which defines the hollow interior chamber 120, the upper and lower vertebral engaging surfaces 122, 124, and the bone growth openings 126. A bone growth material (not shown) may be positioned within the hollow interior chamber 120 to promote bone growth from the adjacent vertebrae and into the hollow interior chamber 120 via the bone growth openings 126.

Similar to the anchor device 14 associated with the spinal implant 10, the anchor device 114 is at least partially formed of a bioresorbable material which may be selected from any of the materials set forth above with regard to the anchor device 14. Additionally, the anchor device 114 may be provided with any of the elements or features associated with the embodiments set forth above with regard to the anchor device 14. In one embodiment, the anchor device 114 is engagable with the adjacent vertebrae and is configured to temporarily secure the interbody device 112 to the adjacent vertebrae for a period of time sufficient to facilitate bone growth therebetween, and to temporarily rigidly secure the interbody device 112 to the adjacent vertebrae to substantially prevent relative movement therebetween to facilitate bone growth. Bone growth occurs through the bone growth openings 126 in the interbody device 112 and preferably into and through the hollow interior chamber 120. Subsequent to such bone growth, at least a portion of the anchor device 114 is resorbed or absorbed into the body and the force originally exerted onto the interbody device 112 to secure the interbody device 112 in position relative to the adjacent vertebrae is removed.

In the illustrated embodiment, the anchor device 114 is configured as an elongate member 130 formed of the resorbable material and having a length l sized to span a distance between the adjacent vertebrae. The elongate member 130 includes a first end portion 130a opposite a second end portion 130b with an intermediate portion 130c disposed between the end portions 130a, 130b. The intermediate portion 130c of the elongate member 130 is coupled with the interbody device 112 via a threaded interconnection 136. The threaded interconnection 136 comprises first and second internally threaded passages 138a, 138b formed in the interbody device 112, with each of the passages 138a, 138b configured to matingly receive and engage respective threaded fasteners 140a, 140b extending through the intermediate portion 130c of the elongate member 130. The threaded fasteners 140a, 140b may be at least partially formed of a bioresorbable material configured for adsorption or resorbtion into the body subsequent to bone ingrowth into the interbody device 112. In an alternative embodiment, the elongate member 130 may include one or more recessed or countersunk regions for receiving the screw head portions 142a, 142b to reduce the profile of the spinal implant 110 and minimize the risk of damaging or irritating surrounding tissue or anatomic structures. In the illustrated embodiment, the interbody device 112 remains coupled with the anchor device 114 attached to the upper and lower vertebrae until sufficient bone growth has occurred to prevent the interbody device 112 from moving or becoming dislodged from between the upper and lower vertebrae.

While one particular type and configuration of a threaded interconnection 136 has been illustrated and described herein, it should also be understood that other interconnection configurations are contemplated for use in association with the present invention. For example, in an alternative embodiment, the interbody device 112 may include one or more threaded stems extending therefrom and through one or more corresponding passages in the anchor device 114 to be engaged with one or more internally threaded engaging members, such as nuts. Additionally, although the illustrated and described embodiments of the threaded interconnection 136 include two threaded fasteners, it should be appreciated that in alternative embodiments, the threaded interconnection 136 may include only one threaded fastener or may include three or more threaded fasteners. It should further be appreciated that in other embodiments, one or more components of the threaded interconnection 136 may be at least partially formed of a resorbable material as described herein. Additionally, although not specifically illustrated in FIG. 3, it should be understood that each end of the elongate member 130 is engaged to an adjacent vertebra by an anchoring member, including one or more bone screws, tacks, bolts, pins, prongs, hooks, anchors or any other suitable bone engaging member known to those of skill in the art. In one non-limiting form, at least a portion of each anchoring member is formed of a bioresorbable material.

Referring now to FIG. 4, shown therein is a spinal implant 210 according to another form of the present invention. The spinal implant 210 generally includes an interbody device 212 sized and shaped to be positioned within an intervertebral disc space between adjacent vertebrae, and an anchor device 214 at least partially formed of a bioresorbable material and configured to temporarily secure the interbody device 212 to the adjacent vertebrae for a period of time sufficient to facilitate bone growth therebetween.

In many ways, the interbody device 212 is configured similar to the interbody device 112 illustrated and described above with regard to the spinal implant 110, including a height which substantially corresponds to the natural disc space height between adjacent vertebrae, and a depth and a width which substantially correspond to the outer profile of the natural vertebral endplates such that the interbody device 212 substantially fills the intervertebral disc space between the adjacent vertebrae. The interbody device 212 may be formed from any of the materials set forth above with regard to the interbody device 12, and may be provided with any of the elements or features associated with the embodiments set forth above with regard to the interbody devices 12 and 112. In the illustrated embodiment, the interbody device 212 is configured as a fusion device that promotes fusion between the adjacent vertebrae, and includes a hollow interior chamber 220 and upper and lower vertebral engaging surfaces 222, 224 configured for engagement with the endplates of adjacent vertebrae. Each of the upper and lower vertebral engaging surfaces 222, 224 defines at least one bone growth opening 226 extending therethrough and communicating with the hollow interior chamber 220. In the illustrated embodiment, the interbody device 212 includes a continuous outer parametrical wall 228 which defines the hollow interior chamber 220, the upper and lower vertebral engaging surfaces 222, 224, and the bone growth openings 226. A bone growth material (not shown) may be positioned within the hollow interior chamber 220 to promote bone growth from the adjacent vertebrae and into the hollow interior chamber 220 via the bone growth openings 226.

In the illustrated embodiment, the anchor device 214 is configured as an elongate member 230 formed of the resorbable material and having a length l sized to span a distance between the adjacent vertebrae. Additionally, the anchor device 214 may be provided with any of the elements or features associated with the embodiments set forth above with regard to the anchor device 14 or the anchor device 114. In one embodiment, the anchor device 214 is engagable with the adjacent vertebrae and is configured to temporarily secure the interbody device 212 to the adjacent vertebrae for a period of time sufficient to facilitate bone growth therebetween, and to temporarily rigidly secure the interbody device 212 to the adjacent vertebrae to substantially prevent relative movement therebetween to facilitate bone growth.

In the illustrated embodiment, the anchor device 214 includes an upper flange member 230 and a lower flange member 232 which are each at least partially formed of a bioresorbable material. However, in other embodiments, only one of the flanges 230, 232 need by formed of a bioresorbable material. The upper flange member 230 includes a first end portion 230a and an opposite second end portion 230b coupled with the interbody device 212, while the lower flange member 232 similarly includes a first end portion 232a and an opposite second end portion 232b coupled with the interbody device 212. Each of end portions 230a, 232a extend away from a central portion 213 of the interbody device 212 and are separated from one another along the length l of the anchor device 214. In one embodiment, each of the upper and lower flanges 230, 232 are formed entirely of a resorbable material. However, in other embodiments, only portions of the flanges 230, 232 are formed of a resorbable material. For example, in one embodiment, the end portions 230a, 232a may be formed of a resorbable material, with the opposite end portions 230b, 232b formed of a non-resorbable material. In another embodiment, the end portions 230b, 232b may be formed of a resorbable material, with the end portions 230a, 232a formed of a non-resorbable material.

In the illustrated embodiment, the end portions 230b, 232b of the elongate member 230 are formed integral with the interbody device 212. However, it is contemplated that the end portions 230a, 230b may be coupled with the interbody device 212 by a threaded interconnection similar to threaded interconnection 136 illustrated and described above with regard to the spinal implant 110, or may be coupled with the interbody device 212 via other coupling arrangements including adhering, fusing, clamping, pinning, riveting, tacking or any other coupling technique know to those of skill in the art. Although the upper and lower flanges 230, 232 are illustrated as extending in a direction substantially transverse to the interbody device 212, it is also contemplated that one or both of the flanges 230, 232 may extend in other directions relative to the interbody device 212, including at an oblique angle relative to the interbody device 212. Additionally, although not specifically illustrated in FIG. 4, it should be understood that each of the flange members 230, 232 are engaged to an adjacent vertebra by an anchoring member, including one or more bone screws, tacks, bolts, pins, prongs, hooks, anchors or any other suitable bone engaging member known to those of skill in the art. In one non-limiting form, at least a portion of each anchoring member is formed of a bioresorbable material.

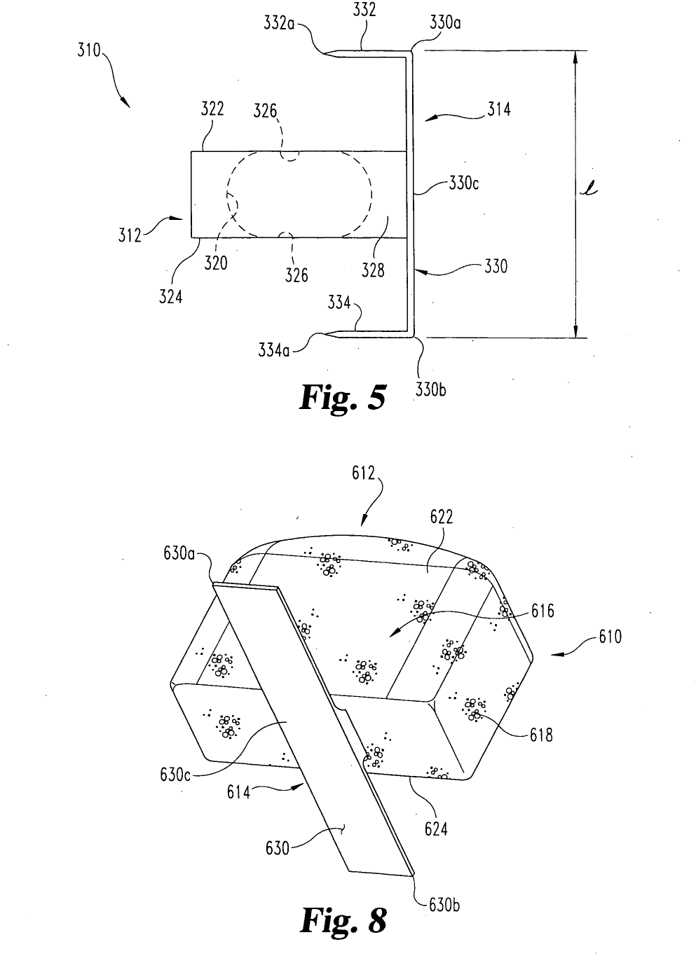

Referring to FIG. 5, shown therein is a spinal implant 310 according to another form of the present invention. The spinal implant 310 generally includes an interbody device 312 sized and shaped to be positioned within an intervertebral disc space between adjacent vertebrae, and an anchor device 314 at least partially formed of a bioresorbable material and configured to temporarily secure the interbody device 312 to the adjacent vertebrae for a period of time sufficient to facilitate bone growth therebetween.

In many ways, the interbody device 312 is configured similar to the interbody devices 112 and 212 illustrated and described above with regard to the spinal implants 110 and 210, including a height which substantially corresponds to the natural disc space height between adjacent vertebrae, and a depth and a width which substantially correspond to the outer profile of the natural vertebral endplates such that the interbody device 312 substantially fills the intervertebral disc space between the adjacent vertebrae. The interbody device 312 may be formed from any of the materials set forth above with regard to the interbody device 12, and may be provided with any of the elements or features associated with the embodiments set forth above with regard to the interbody devices 12, 112 and 212. In the illustrated embodiment, the interbody device 312 is configured as a fusion device that promotes fusion between the adjacent vertebrae, and includes a hollow interior chamber 320 and upper and lower vertebral engaging surfaces 322, 324 configured for engagement with the endplates of adjacent vertebrae. Each of the upper and lower vertebral engaging surfaces 322, 324 defines at least one bone growth opening 326 extending therethrough and communicating with the hollow interior chamber 320. In the illustrated embodiment, the interbody device 312 includes a continuous outer parametrical wall 328 which defines the hollow interior chamber 320, the upper and lower vertebral engaging surfaces 322, 324, and the bone growth openings 326. A bone growth material (not shown) may be positioned within the hollow interior chamber 320 to promote bone growth from the adjacent vertebrae and into the hollow interior chamber 320 via the bone growth openings 326.

In the illustrated embodiment, the anchor device 314 is configured as an elongate member 330 formed of the resorbable material and having a length l sized to span a distance between the adjacent vertebrae. Additionally, the anchor device 314 may be provided with any of the elements or features associated with the embodiments set forth above with regard to the anchor device 14 or the anchor devices 114 and 214. In one embodiment, the anchor device 314 is engagable with the adjacent vertebrae and is configured to temporarily secure the interbody device 312 to the adjacent vertebrae for a period of time sufficient to facilitate bone growth therebetween, and to temporarily rigidly secure the interbody device 312 to the adjacent vertebrae to substantially prevent relative movement therebetween to facilitate bone growth.

In one embodiment of the invention, the elongate member 330 has a staple-like configuration including a first anchoring portion or prong 332 and a second anchoring portion or prong 334. The prongs 332, 334 extend transversely from respective end portions 330a, 330b of a bridge portion 330c defined by the elongate member 330. In one specific embodiment, the prongs 332, 334 are formed integral with the bridge portion 330c to form a single piece, unitary staple-like structure. However, in other embodiments, the prongs 332, 334 may be formed separately from the bridge portion 330c and coupled thereto by any coupling technique known to those of skill in the art. In one embodiment, the bridge portion 330c is formed integral with the interbody device 312 to form a single piece, unitary structure. However, in other embodiments, the bridge portion 330c may be coupled with the interbody device 312 via adhering, fusing, tacking, pinning, clamping, screwing, bolting or any other coupling technique known to those of skill in the art. In one embodiment of the invention, the coupling arrangement is at least partially formed of a resorbable material.

In the illustrated embodiment, each of the prongs 332, 334 are structured for engagement with the adjacent vertebrae and include a pointed or spiked ends 332a, 334a to facilitate introduction or penetration into vertebral bone. Although each of the anchoring portions 332, 334 are illustrated as having a substantially smooth outer surface, it is also contemplated that the prongs 332, 334 may be provided with one or more bone engagement enhancing features, including but not limited to barbs such as may be found on conventional bone staples. Furthermore, the anchor device 314 may include one or more additional prongs extending from the bridge portion 330c of the elongate member 330 in addition to the prongs 332, 334 to provide a multi-pronged staple configuration, including three prongs or four or more prongs.

Although the prongs 332, 334 are illustrated as being arranged substantially perpendicular to the bridge portion 330c, other configurations are contemplated. For example, the prongs 332, 334 may extend in one or more directions toward or away from interbody device 312 to facilitate engagement with the adjacent vertebrae. In one embodiment of the invention, the prongs 332, 334 and the bridge portion 330c are entirely formed of a resorbable material. However, in other embodiments, only one of the prongs 332, 334 and the bridge portion 330c are formed of a bioresorbable material, or select portions thereof are formed of a bioresorbable material. For example, in one embodiment, the bridge portion 330c may be formed of a resorbable material while the prongs 332, 334 are formed of a non-resorbable material. In another embodiment, the prongs 332, 334 are formed of a resorbable material while the bridge portion 330c is formed of a non-resorbable material.

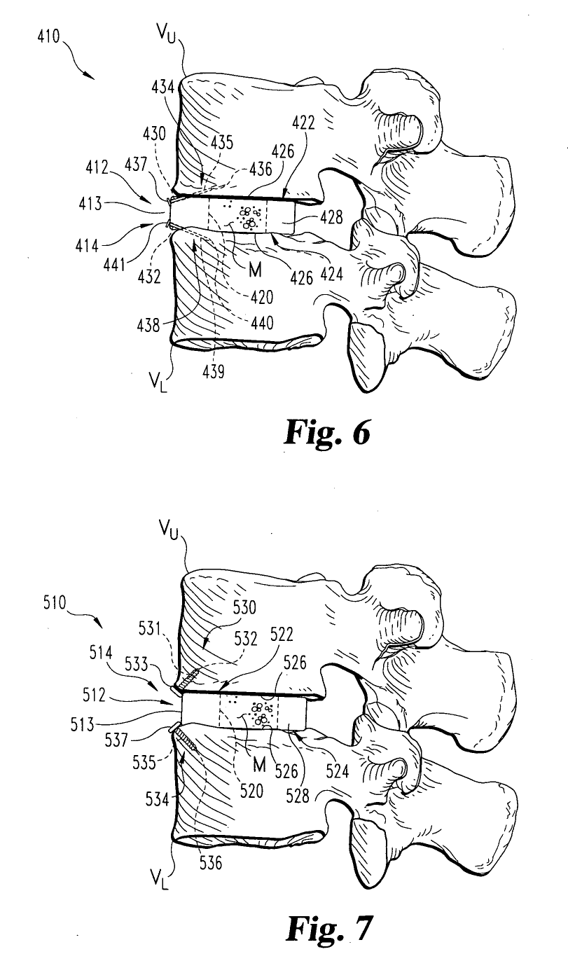

Referring to FIG. 6, shown therein is a spinal implant 410 according to another form of the present invention, as positioned between adjacent upper and lower vertebrae V_{U}, V_{L}. The spinal implant 410 generally includes an interbody device 412 sized and shaped to be positioned within an intervertebral disc space between the upper and lower vertebrae V_{U}, V_{L}, and one or more anchor devices 414 at least partially formed of a bioresorbable material and configured to temporarily secure the interbody device 412 to the adjacent upper and lower vertebrae V_{U}, V_{L} for a period of time sufficient to facilitate bone growth therebetween.

The interbody device 412 is generally sized and shaped for placement within the disc space between the upper and lower vertebrae V_{U}, V_{L} such that the interbody device 412 substantially fills the intervertebral disc space between the adjacent vertebrae V_{U}, V_{L}. It should be appreciated that one or more characteristics of the interbody device 412 may vary in alternative embodiments, as set forth above with regard to the interbody device 12. In the illustrated embodiment, the interbody device 412 is configured as a fusion device that promotes fusion between the upper and lower vertebrae V_{U}, V_{L}, including a hollow interior chamber 420 and upper and lower vertebral engaging surfaces 422, 424 configured for engagement with endplates of the upper and lower vertebrae V_{U}, V_{L}. Each of the upper and lower vertebral engaging surfaces 422, 424 defines at least one bone growth opening 426 extending therethrough and communicating with the hollow interior chamber 420. In one embodiment, the upper and lower vertebral engaging surfaces 422, 424 are substantially flat and smooth. However, in other embodiments, the vertebral engaging surfaces 422, 424 may be provided with a plurality of surface features that engage the vertebral endplates to inhibit movement of the interbody device, including, for example, teeth or peeks, ridges, grooves or valleys, recessed regions, spikes, surface roughening, or other suitable surface features that would occur to one of skill in the art. In the illustrated embodiment, the interbody device 412 includes a continuous outer parametrical wall 428 which defines the hollow interior chamber 420, the upper and lower vertebral engaging surfaces 422, 424, and the bone growth openings 426. The interbody device 412 further includes a bone growth material M positioned within the interior chamber 420 to promote bone growth from the upper and lower vertebrae V_{U}, V_{L} and into the hollow interior chamber 420 via the bone growth openings 426, as described above with regard to the spinal implant 10.

In the illustrated embodiment of the interbody device 412, the parametrical wall 428 or the interbody device 412 defines a first passage 430 opening at and extending from the end wall 413 to an opening extending through the upper vertebral engaging surface 422, and a second passage 432 opening at and extending from the end wall 413 to an opening extending through the lower vertebral engaging surface 424. The passages 430, 432 are generally configured and arranged to receive bone engaging members 434, 438 to engage the interbody device 412 to the upper and lower vertebrae V_{U}, V_{L}. In the illustrated embodiment, the bone engaging members 434, 438 are generally configured as bone nails or screws, with each bone engaging member including a stem portion 435, 439 and a pointed tip portion 436, 440 opposite an enlarged head portion 437, 441. It should be appreciated that as the stem portions 435, 439 are engaged with the respective vertebrae V_{U}, V_{L}, the head portions 437, 441 come into abutting contact with the end wall 413 of the interbody device 412 to secure the device 412 in position relative to the adjacent vertebrae V_{U}, V_{L}. In a non-illustrated embodiment of the invention, the end wall 413 may include one or more recessed or countersunk portions configured to receive the screw head portions 437, 441 to reduce the overall profile of the spinal implant 410 and the possibility of interference with adjacent spinal tissue or other anatomic features. In other non-illustrated embodiments of the invention, the bone engaging members 434, 438 may be integrally formed with the interbody device 412 to form a unitary, single piece spinal implant 410. It should be appreciated that in such embodiments, the bone engaging members 434, 438 will contact and engage the upper and lower vertebrae as the interbody device 412 is inserted into the disc space between the vertebrae.

Although the stem portions 435, 439 are illustrated as having a substantially smooth exterior surface, it should be appreciated that one or more exterior surface features may be provided to enhance engagement between the bone engaging members and the vertebrae, including but not limited to external threads, barbs or other suitable surface features that would occur to one of skill in the art. Furthermore, it is contemplated that the bone engaging members 434, 438 may be configured as screws, tacks, bolts, pins, prongs, hooks, anchors or any other suitable bone engaging member known to those of skill in the art. In one embodiment of the invention, the bone engaging members 434, 438 are entirely formed of a resorbable material. In other embodiments, only a portion of one or more of the bone engaging members 434, 438 are formed of a resorbable material. For example, in one specific embodiment, the head portions 437, 441 may be formed of a resorbable material while all or part of the stem portions 435, 439 are formed of a non-resorbable material.

Referring to FIG. 7, shown therein is a spinal implant 510 positioned between adjacent upper and lower vertebrae V_{U}, V_{L}. The spinal implant 510 generally includes an interbody device 512 sized and shaped to be positioned within an intervertebral disc space between the adjacent upper and lower vertebrae V_{U}, V_{L}, and an anchor device 514 at least partially formed of a bioresorbable material and configured to temporarily secure the interbody device 512 to the adjacent upper and lower vertebrae V_{U}, V_{L} for a period of time sufficient to facilitate bone growth therebetween.

The interbody device 512 is configured similar to the interbody device 412 illustrated and described above. The interbody device 512 is generally sized and shaped for placement in the disc space between the upper and lower vertebrae V_{U}, V_{L} such that the interbody device 512 substantially fills the intervertebral disc space between the adjacent vertebrae V_{U}, V_{L}. It should be appreciated that one or more characteristics of the interbody device 512 may vary in alternative embodiments, as set forth above in regard to interbody device 12. Furthermore, interbody device 512 is configured as a fusion device that promotes fusion between the upper and lower vertebrae V_{U}, V_{L}, including a hollow interior chamber 520 and upper and lower vertebral engaging surfaces 522, 524 configured for engagement with endplates of the upper and lower vertebrae V_{U}, V_{L}. Each of the upper and lower vertebral engaging surfaces 522, 524 defines at least one bone growth opening 526 extending therethrough and communicating with the hollow interior chamber 520. In the illustrated embodiment, the interbody device 512 includes a continuous outer parametrical wall 528 which defines the hollow interior chamber 520, the upper and lower vertebral engaging surfaces 522, 524, and the bone growth openings 526. Furthermore, the interior chamber 520 includes a bone growth material M to promote bone growth from the upper and lower vertebrae V_{U}, V_{L} into the hollow interior chamber 520 via the bone growth openings 526.

The anchor device 514 generally includes a first bone engaging member 530 and a second bone engaging member 534. However, it is contemplated that the anchor device 514 may include only one bone engaging member or may include three or more bone engaging members. In the illustrated embodiment, the bone engaging members 530, 534 are configured as bone screws, each including a longitudinal threaded stem portion 531, 535 disposed between a leading tip 532, 536 and a head portion 533, 537. The stem portions 531, 535 are generally configured to threadingly engage the upper and lower vertebrae V_{U}, V_{L} at a position adjacent to the interbody device 512, and can be provided with cutting flutes or other structures that provide the bone engaging members 530, 534 with self-tapping and/or self-drilling capabilities.

It should be appreciated that as the threaded stem portions 531, 535 are engaged with the respective vertebrae V_{U}, V_{L} at the prepared location adjacent the interbody device 512, the head portions 533, 537 come into abutting contact with the end wall 513 of the interbody device 512 to secure the interbody device 512 in position between the adjacent vertebrae V_{U}, V_{L}. In the illustrated embodiment, the head portions 533, 537 are engaged against upper and lower corner portions or edges defined between the end wall 513 and the upper and lower vertebral engaging surfaces 522, 524. Although the illustrated embodiment of the interbody device 512 does not include any structural features for receiving the head portions 533, 537 of the bone screws 530, 534 therein, it is contemplated that the end wall 513 and/or the vertebral engaging surfaces 522, 524 may be provided with recessed regions or countersunk regions to receive at least a portion of the heads 533, 537 therein. Furthermore, it is contemplated that the bone engaging members 530, 534 may be configured as tacks, bolts, pins, prongs, hooks, anchors or any other suitable bone engaging member known to those of skill in the art. In one embodiment of the invention, the bone engaging members 530, 534 are formed entirely of a resorbable material. However, in other embodiments of the invention, only a portion of one or more of the bone engaging members 530, 534 are formed of a resorbable material. For example, in one specific embodiment, the head portions 533, 537 may be formed of a resorbable material while all or part of the stem portions 531, 535 are formed of a non-resorbable material.

Referring to FIG. 8, shown therein is a spinal implant 610 according to another form of the present invention. The spinal implant 610 generally includes the same elements and features illustrated and described above with regard to the spinal implant 10, including an interbody device 612 and an anchor device 614. The interbody device 612 includes upper and lower vertebral engaging surfaces 622, 624 configured for engagement with the vertebral endplates of adjacent upper and lower vertebrae. In the illustrated embodiment, the anchor device 614 is configured as an elongate member 630 including end portions 630a, 630b for anchoring to adjacent upper and lower vertebrae, and an intermediate portion coupled with the interbody device 612. However, unlike the interbody device 12 which includes a hollow interior 20, the interbody device 612 is formed of a material defining pores 618 to facilitate bone in-growth, but being provided with a substantially solid configuration (i.e., does not define a hollow interior). In one embodiment, the interbody device 612 is formed of a porous, high strength material providing a substantially solid body 616 defining interconnected pores 618. In one embodiment, the interbody device is formed of a porous tantalum-carbon composite, as described in U.S. Pat. No. 5,282,861 to Kaplan, the contents of which are incorporated herein by reference. However, other materials are also contemplated as falling within the scope of the present invention. Additionally, as indicated above, other types of interbody devices are also contemplated for use in association with the present invention including, for example, motion preserving devices such as artificial discs and articulating implants, and expandable devices including expandable interbody implants and corpectomy implants.

While the invention has been illustrated and described in detail in the drawings and foregoing description, the same is to be considered as illustrative and not restrictive in character, it being understood that only the preferred embodiments have been shown and described and that all changes and modifications that come within the spirit of the invention are desired to be protected.

What is claimed is:

1. A spinal implant, comprising:
an interbody device sized and shaped to be positioned within an intervertebral disc space between adjacent upper and lower vertebrae; and
an anchor device at least partially formed of a resorbable material, said anchor device coupled to said interbody device and engaged to the upper and lower vertebrae, said anchor device configured to temporarily secure said interbody device to the upper and lower vertebrae for a period of time sufficient to facilitate bone growth onto and/or through the interbody device.

2. The spinal implant of claim 1, wherein said anchor device is configured to temporarily rigidly secure said interbody device to the upper and lower vertebrae to substantially prevent relative movement therebetween to facilitate said bone growth.

3. The spinal implant of claim 1, wherein said anchor device comprises an elongate member formed of said resorbable material and having a length sized to span a distance between said upper and lower vertebrae, said elongate member anchored to the upper and lower vertebrae by at least two bone engaging members.

4. The spinal implant of claim 3, wherein said bone engaging members comprise bone screws.

5. The spinal implant of claim 3, wherein said bone engaging members are each formed of a resorbable material.

6. The spinal implant of claim 1, wherein said interbody device is formed of a substantially rigid material.

7. The spinal implant of claim 6, wherein said interbody device is formed of a non-resorbable material.

8. The spinal implant of claim 1, wherein said interbody device comprises a fusion device defining a hollow interior chamber and including upper and lower vertebral engaging surfaces configured for engagement with endplates of the upper and lower vertebrae, each of said upper and lower vertebral engaging surfaces defining at least one opening extending therethrough and communicating with said hollow interior chamber.

9. The spinal implant of claim 8, further comprising a bone growth material positioned within said hollow interior chamber to promote bone growth from the upper and lower vertebrae and into said hollow interior chamber of said fusion device.

10. The spinal implant of claim 1, wherein said interbody device comprises an artificial disc configured to provide articulating motion between the upper and lower vertebrae.

11. The spinal implant of claim 1, wherein said anchor device comprises at least two bone engaging members each formed of said resorbable material, a first of said bone engaging members coupled with an upper portion of said interbody device and engaged with said upper vertebra, a second of said bone engaging members coupled with a lower portion of said interbody device and engaged with said lower vertebra.

12. The spinal implant of claim 11, wherein said bone engaging members comprise bone screws, each of said bone screws extending through respective openings in said upper and lower portions of said interbody device and into engagement with vertebral bone to temporarily secure said interbody device to the upper and lower vertebrae.

13. The spinal implant of claim 1, wherein said anchor device is formed integral with said interbody device to form a unitary, single-piece structure.

14. The spinal implant of claim 1, wherein said anchor device is coupled to said interbody device by a fastener, said fastener formed of a resorbable material.

15. A spinal implant, comprising:
an interbody device sized and shaped to be positioned within an intervertebral disc space between adjacent upper and lower vertebrae; and
an elongate member formed of a resorbable material and having a length sized to span a distance between the upper and lower vertebrae, said elongate member coupled to said interbody device and having first and second end portions, said first end portion anchored to the upper vertebra by a first bone engaging member, said second end portion anchored to the lower vertebra by a second bone engaging member, said elongate member configured to temporarily secure said interbody device to the upper and lower vertebrae for a period of time sufficient to facilitate bone growth onto and/or through the interbody device.

16. The spinal implant of claim 15, wherein said elongate member is configured to temporarily rigidly couple said interbody device to the upper and lower vertebrae to substantially prevent relative movement therebetween to facilitate said bone growth.

17. The spinal implant of claim 15, wherein said elongate member comprises a plate including said first and second end portions and an intermediate portion therebetween, said intermediate portion coupled with said interbody device.

18. The spinal implant of claim 15, wherein said first end portion comprises an upper flange portion extending from an upper portion of said interbody device for positioning adjacent the upper vertebra; and
wherein said second end portion comprises a lower flange portion extending from a lower portion of said interbody device for positioning adjacent the lower vertebra.

19. The spinal implant of claim 15, wherein said first and second end portions of said elongate member each define at least one opening extending therethrough and sized to receive respective ones of said first and second bone engaging members therethrough and into engagement with vertebral bone.

20. The spinal implant of claim 19, wherein said bone engaging members comprise bone screws.

21. The spinal implant of claim 19, wherein said bone engaging members comprise bone tacks.

22. The spinal implant of claim 15, wherein said bone engaging members are each formed of a resorbable material.

23. The spinal implant of claim 15, wherein said elongate member comprises a staple including a bridge portion and prong portions formed integral with opposite end portions of said bridge portion and configured to penetrate vertebral bone to temporarily secure said interbody device to the upper and lower vertebrae to facilitate said bone growth.

24. The spinal implant of claim 15, wherein said interbody device is formed of a non-resorbable metallic material.

25. The spinal implant of claim 15, wherein said interbody device comprises a fusion device defining a hollow interior chamber and including upper and lower vertebral engaging surfaces configured for engagement with endplates of the upper and lower vertebrae, each of said upper and lower vertebral engaging surfaces defining at least one opening extending therethrough and communicating with said hollow interior chamber.

26. The spinal implant of claim 15, wherein said interbody device comprises an artificial disc configured to provide articulating motion between the upper and lower vertebrae.

27. The spinal implant of claim 15, wherein said interbody device comprises a pair of fusion devices bilaterally positioned within the intervertebral disc space.

28. The spinal implant of claim 15, wherein said elongate member is formed integral with said interbody device to form a unitary, single-piece structure.

29. The spinal implant of claim 15, wherein said elongate member is coupled to said interbody device by a fastener, said fastener formed of said resorbable material.

30. A method of vertebral interbody fusion, comprising:
providing an interbody device and an anchor device at least partially formed of a resorbable material;
positioning the interbody device within an intervertebral disc space between adjacent upper and lower vertebrae;
coupling the anchor device with the interbody device;
engaging the anchor device to the upper and lower vertebrae; and
wherein the anchor device temporarily secures the interbody device to the upper and lower vertebrae for a period of time sufficient to facilitate bone growth onto and/or through the interbody device.

31. The method of claim 30, wherein the temporarily securing substantially prevents relative movement between the interbody device and the upper and lower vertebrae to facilitate the bone growth.

32. The method of claim 30, wherein the anchor device comprises an elongate member formed of the resorbable material and having a length sized to span a distance between the upper and lower vertebrae.

33. The method of claim 32, wherein the elongate member has first and second end portions and an intermediate portion; and
wherein the coupling comprises attaching the intermediate portion to the interbody device; and
wherein the engaging comprises anchoring the first end portion of the elongate member to the upper vertebra by a first bone engaging member and anchoring the second end portion of the elongate member to the lower vertebra by a second bone engaging member.

34. The method of claim 33, wherein the attaching of the intermediate portion to the interbody device comprises fastening the intermediate portion with the interbody device.

35. The method of claim 33, wherein the attaching of the intermediate portion to the interbody device comprises integrally forming the intermediate portion with the interbody device.

36. The method of claim 33, wherein the bone engaging members are each formed of the resorbable material.

37. The method of claim 32, wherein the elongate member comprises upper and lower flange portions extending from the interbody device;
wherein the coupling comprises attaching the upper flange portion to an upper portion of the interbody device and attaching the lower flange portion to a lower portion of the interbody device; and
wherein the engaging comprises anchoring the upper flange portion to the upper vertebra by a first bone engaging member and anchoring the lower flange portion to the lower vertebra by a second bone engaging member.

38. The method of claim 37, wherein the attaching of the upper and lower flange portions with the upper and lower portions of the interbody device comprises integrally forming the upper and lower flange portions with the upper and lower portions of the interbody device.

39. The method of claim 37, wherein the bone engaging members are each formed of the resorbable material.

40. The method of claim 30, wherein the interbody device comprises a fusion device defining a hollow interior chamber and including upper and lower vertebral engaging surfaces configured for engagement with endplates of the upper and lower vertebrae, each of said upper and lower vertebral engaging surfaces defining at least one opening extending therethrough and communicating with the hollow interior chamber; and
further comprising inserting a bone growth material within the hollow interior chamber to promote bone growth from the upper and lower vertebrae and into the hollow interior chamber of the fusion device.

41. The method of claim 30, wherein the interbody device comprises an artificial disc configured to provide articulating motion between the upper and lower vertebrae; and
wherein the anchor device temporarily secures the artificial disc to the upper and lower vertebrae for a period of time sufficient to facilitate bone growth with the artificial disc.

42. The method of claim 30, wherein the anchor device comprises at least two bone engaging members each formed of the resorbable material; and
wherein the coupling and the engaging comprise coupling a first of the bone engaging members with an upper portion of the interbody device and engaging the first bone engaging member with the upper vertebra, coupling a second of the bone engaging members with a lower portion of the interbody device and engaging the second bone engaging member with the lower vertebra.

43. The method of claim 42, wherein the bone engaging members comprise bone screws, each of the bone screws extending through respective openings in the upper and lower portions of the interbody device and into engagement with vertebral bone to temporarily secure the interbody device to the upper and lower vertebrae.
